(19) United States
(12) Patent Application Publication
(10) Pub. No.: US 2011/0144748 A1
(43) Pub. Date: Jun. 16, 2011

(54) STRUCTURE OF BREAST AUGMENTATION POCKET

(75) Inventors: YAU-ZEN CHANG, TAOYUAN COUNTY (TW); MOU-CHUAN LIU, TAIPEI CITY (TW)

(73) Assignee: CHANG GUNG UNIVERSITY, TAOYUAN COUNTY (TW)

(21) Appl. No.: 12/638,547

(22) Filed: Dec. 15, 2009

(51) Int. Cl. A61F 2/12

(52) U.S. Cl. 623/8

(57) ABSTRACT

A structure of breast augmentation pocket comprises a biocompatible liquid pocket, wherein a biocompatible liquid and a plurality of hollow silicone gel particles in the forms of large, small circles, or various shapes are filled inside. The silicone gel is applied with colors to form recognizable silicone gel such that the silicone gel particles are readily recognized and then cleaned away in case of rupture of the biocompatible liquid pocket. The silicone gel particles are hollow with the same average density as that of the biocompatible liquid so as to be suspended in the biocompatible liquid, whereby a touch feeling more similar to that of real tissue is provided because of the combination of biocompatible liquid with silicone gel particles. With good biocompatibility with non-dissolvability, the silicone gel particles neither lead to immune reactions nor cause any possible sequelae.

### FIELD OF THE INVENTION

The present invention relates to the technical field of breast augmentation pockets, particularly to a structure of breast augmentation pocket.

### BACKGROUND

Breast augmentation is a plastic surgery used for improving women's figure. The breast augmentation surgery first appeared in 1960s. In this surgery, some fillers are placed in the breast. One of the materials for breast augmentation is “silicone gel”, which provides soft touch and feel. Yet, leakage of silicone gel from the artificial breast pocket happens and may cause formation of lumps and chronic inflammation within breast tissues. In the early 1980s, researches in America proposed the doubt that silicone gel may cause breast cancer. Thus, American authorities once prohibited the use of silicone gel, while physiological saline with higher safety was used instead. Even if there is rupture or leakage, the saline solution absorbed by the human body will not impact a patient's health.

For patients of silicon gel breast reconstruction surgery, there is nothing more important than a natural and soft feel after the surgery. For the cosmetic surgeon, the most technical challenge is the so-called “total implantation.” When the artificial breast pocket is implanted into the human body, a substantially bulky oval ball has to be forcibly squeezed through the cut in the outer skin. This is greatly different from the process of “staged implantation” used for the artificial breast surgery with saline pocket in the past, in which the artificial breast pocket is rolled as a cigar-like shape firstly so as to be implanted into the human body, and physiological saline is then poured in to expand the artificial breast pocket. The length of the cut required for the artificial breast surgery with saline pocket (1-2 cm) is much shorter than that for the artificial breast surgery of silicone gel (5-8 cm) no matter where the surgery cut is (armpit, lower edge of breast, lower edge of areola).

The conventional saline pocket has bad touch feeling. The surface layer of the conventional saline pocket is a layer of silicone gel, which does harm the human body.

Thus, to develop an innovative structure with ideal practicality in order to solve the problems existed in the above conventional structure is expected.

Technical problems to be solved are described as follows. The shape of the conventional silicone gel is not fixed after rupture; thus, it is difficult to remove it when a rupture happens. As a result, the silicone gel remained in the human body may cause anxiety of cancer risk and pathological changes. Besides, it requires a need for a larger cut in the surgical operation. When the conventional saline pocket is ruptured, the leaked physiological saline is absorbed by the human body. Nevertheless, in order to maintain appropriate resilience, the saline pocket is usually not full when it is implanted. In such a way, wrinkles may show at its periphery. If fatty tissue of the patient is too thin to firmly cover the implanted artificial breast pocket, wrinkles at the periphery of the breast can be felt. Moreover, the touch feeling of the saline pocket is not good, and the implantation position is deeper, which is more painful.

In view of this, the present invention contemplates functions for breast augmentation pockets by improving its practicality through a new structure of breast augmentation pocket.

### SUMMARY OF THE INVENTION

One objective of the present invention is to provide a structure of breast augmentation pocket comprising a biocompatible liquid pocket, wherein a biocompatible liquid and a plurality of silicone gel particles in the form of large, small circles, or various shapes are filled. The silicone gel particles are hollow with the same average density as that of the biocompatible liquid so as to be suspended in the biocompatible liquid. The silicone gel is applied with colors, whereby the biocompatible liquid pocket mixes biocompatible liquid with silicon gel particles. Compared with the traditional saline pocket filled with physiological saline only, the present biocompatible liquid pocket provides with a touch feeling more similar to that of real human tissue. The wrinkles possibly generated at the periphery are also avoided by filling the biocompatible liquid pocket with the biocompatible liquid and the silicone gel particles. In case of rupture of the biocompatible liquid pocket, the silicone gel will not lead to immune reactions owing to its good biocompatibility with non-dissolvability. Moreover, in case of a rupture of the biocompatible liquid pocket, the particles therein are readily recognized owing to their colors, and then washed away from or taken out of the human body by means of outside influence more easily.

The surface layer of the conventional saline pocket is a layer of silicone gel, and the silicone gel will not harm the human body. The silicone gel used in the present invention is the silicone gel used for the surface layer of the saline pocket, in such a way the silicone gel particles are safe.

The way for the insertion of the breast augmentation pocket of the present invention is the same as that of the saline pocket, in which only auxiliary means including a cylindrical body and a pusher bar are required for carrying out the implantation of the artificial breast pocket without any special tools.

Technical effects in comparison with those provide by prior art will be described as follows. The structure of the breast augmentation pocket of the present invention provides with a touch feeling more similar to that of real human tissue because of the combination of biocompatible liquid with silicone gel particles. Thus, it can be placed in the superficial layer of skin with less pain accordingly. Moreover, since the biocompatible liquid pocket is provided at the outer layer, the biocompatible liquid therein can be absorbed by the human body if it is ruptured. The silicone gel particles may not lead to immune reactions owing to their good biocompatibility with non-dissolvability, while the colored particle-like gel is also readily recognized and then washed away from or taken out of the human body.

### BRIEF DESCRIPTION OF DRAWINGS

FIG. 1 is a cross section diagram of one embodiment of the present invention.

FIG. 2 is a cross section diagram of silicone gel particles of one embodiment of the present invention.

FIG. 3 is a diagram showing the application of the present invention to the breast.

### DETAILED DESCRIPTION

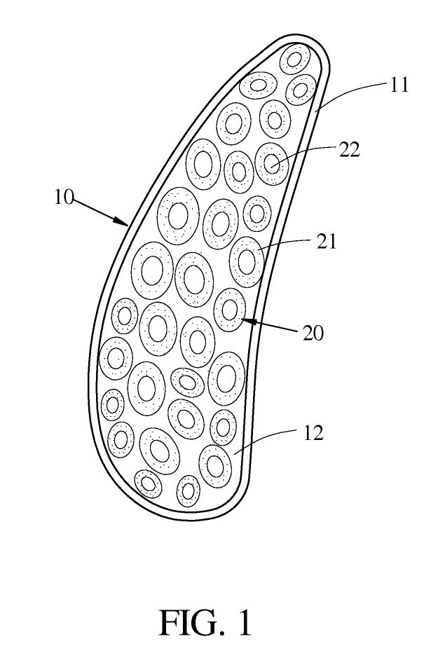

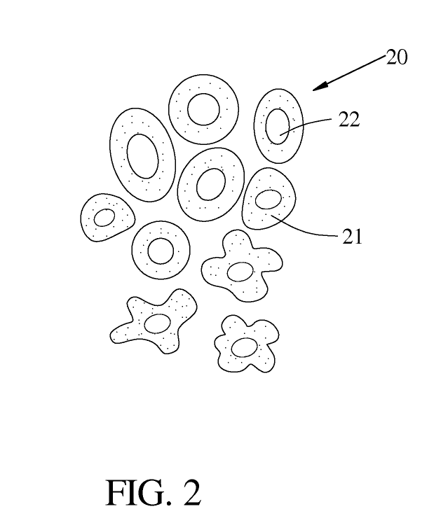

Referring FIGS. 1 and 2, they show a structure of the breast augmentation pocket of the present invention, comprising:

a biocompatible liquid pocket 10 including a surface layer 11, inside which fills with a biocompatible liquid 12, the biocompatible liquid 12 can be physiological saline; and a plurality of silicone gel particles 20 in the form of large, small circles, or various shapes, the silicone gel particles 20 of various shapes being hollow with the same average density as that of the biocompatible liquid 12 so as to be suspended in the biocompatible liquid 12, while each of the silicone gel particles 20 of various shapes being composed of a silicone gel 21 and a hollow portion 22 therein, the silicone gel 21 possibly being applied with colors to form the colored silicone gel particles 20, whereby the biocompatible liquid pocket 10 having the silicone gel particles 20 therein provides with a touch feeling more similar to that of real tissue, because of the combination of biocompatible liquid with silicone gel particles. In case of rupture of the biocompatible liquid pocket 10, the silicone gel particles 20 therein are readily recognized owing to their colors, and then washed away from or taken out of the human body by means of outside influence. For the silicone gel, additionally, due to its good biocompatibility with non-dissolvability, neither immune reactions nor possible sequelae may be caused.

Referring to FIG. 2, the silicone gel particles 20 are formed in the form of large, small circles, or various shapes. Each of the silicone gel particles 20 is provided with a silicone gel 21 and a hollow portion 22 therein. The silicone gel 21 can be further applied with colors to be easily recognized, such that even if the silicone gel 21 flows out of the biocompatible liquid pocket 10 due to rupture, the surgeon can readily recognize the silicone gel 21 with the aid of colors and then clean it away easily by washing it away from the human body or taking it out.

The plurality of silicone gel particles 20 are shaped in the form of large, small circles accommodated within the biocompatible liquid pocket 10.

The plurality of silicone gel particles 20 are presented in the form of various geometrical shapes accommodated within the biocompatible liquid pocket 10.

The plurality of silicone gel particles 20 are presented in the form of various amoeba-like shapes accommodated within the biocompatible liquid pocket 10.

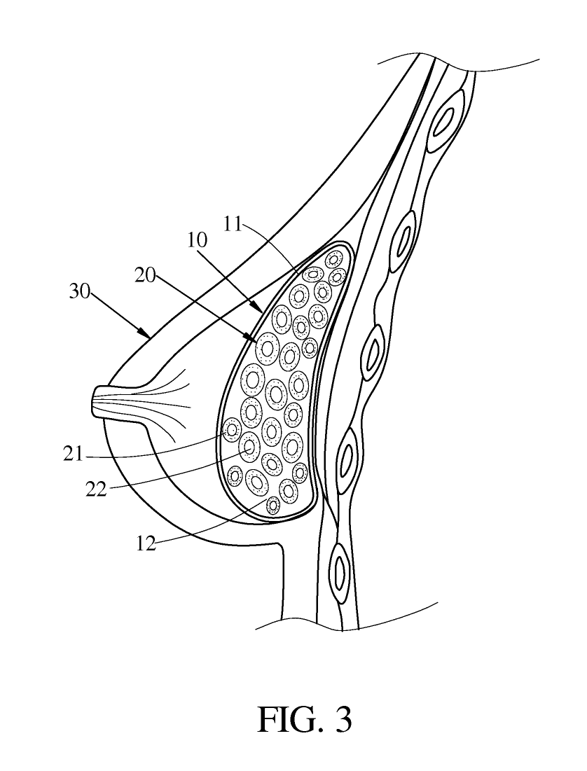

Referring to FIG. 3, the structure of the breast augmentation pocket comprises: a biocompatible liquid pocket 10 composed of a surface layer 11 and an inner layer being positioned inside the surface layer 11 and allowing for accommodating a biocompatible liquid 12 and a plurality of silicone gel particles 20 in the form of large, small circles, or various shapes. The average density of the hollow silicone gel particles 20 is the same as that of the biocompatible liquid 12, so that the silicone gel particles 20 are suspended in the biocompatible liquid 12. Further, each of the silicone gel particles 20 of various shapes is composed of a silicone gel 21 and a hollow portion 22 therein, and the silicone gel 21 can be also applied with colors to form a colored silicone gel 21. The breast augmentation pocket of the present invention provides with a touch feeling more similar to that of real tissue, combining of the biocompatible liquid with the silicone gel particles. In such a way, it can be implanted into a breast 30 in a more superficial layer thereof with less pain accordingly, primarily due to the fact that the biocompatible liquid pocket 10 having the silicone gel particles 20 therein provides with a touch feeling more similar to that of real tissue, combining the biocompatible liquid with the silicone gel particles. Furthermore, in case of a rupture of the biocompatible liquid pocket 10, immediate treatment can be carried out as soon as possible. The silicone gel particles 20 are non-dissolvable and also quite easy to be treated. In addition, in case of the rupture of the biocompatible liquid pocket 10, the silicone gel particles therein can be readily recognized owing to their colors, and then washed away from the human body or taken out by means of outside influence.

The surface layer of the conventional saline pocket is a layer of silicone gel. With good biocompatibility and non-dissolvability, the silicon gel of the layer will not be absorbed by the human body, thus, no immune reactions will occur. The silicone gel used in the present invention is the silicone gel used for the surface layer of the saline pocket. This silicone gel is a silicone gel without being absorbed by the human body with good biocompatibility and non-dissolvability, thus, immune reactions will not occur.

The way for the insertion of the breast augmentation pocket of the present invention is the same as that of the saline pocket, in which only auxiliary means including a cylindrical body and a pusher bar are required for carrying out the implantation of the artificial breast without any special tools.

1. A structure of breast augmentation pocket, comprising:
a biocompatible liquid pocket, including a surface layer and an inner layer being positioned inside said surface layer and allowing for accommodating a biocompatible liquid; and
a plurality of silicone gel particles, provided in said biocompatible liquid within said inner layer of said biocompatible liquid pocket, said silicon gel particles are hollow with a same average density as that of said biocompatible liquid so as to be suspended in said biocompatible liquid, each of said plurality of silicone gel particles being composed of a silicone gel and a hollow portion therein, said plurality of silicone gel particles being easy to be treated if said biocompatible liquid pocket therein is ruptured.

2. The structure of breast augmentation pocket according to claim 1, wherein said plurality of silicone gel particles are shaped in forms of large, small circles accommodated within said biocompatible liquid pocket.

3. The structure of breast augmentation pocket according to claim 1, wherein said silicone gel is a transparent silicone gel.

4. The structure of breast augmentation pocket according to claim 1, wherein said silicone gel is applied with colors so as to form a recognizable silicone gel, such that said silicone gel particles are readily recognized and then cleaned away owing to their colors in case of rupture of said biocompatible liquid pocket.

5. The structure of breast augmentation pocket according to claim 1, wherein said biocompatible liquid is physiological saline.
